(19) United States
(12) Patent Application Publication
(10) Pub. No.: US 2023/0398340 A1
(43) Pub. Date: Dec. 14, 2023

(54) Method and Device for Arresting Epidemics and Pandemics

(71) Applicant: Vasilii Khabuzov, Saint Petersburg (RU)

(72) Inventor: Vasilii Khabuzov, Saint Petersburg (RU)

(21) Appl. No.: 18/034,058

(22) PCT Filed: May 10, 2021
(86) PCT No.: PCT/RU2021/050123

§ 371 Date: Apr. 26, 2023

(30) Foreign Application Priority Data
Oct. 30, 2020 (RU) 2020135840

(51) Int. Cl. A61M 35/00; A61M 11/00; A01N 25/06; A01N 59/16; A01P 1/00

(52) U.S. Cl. CPC A61M 35/20; A61M 11/00; A01N 25/06; A01N 59/16; A01P 1/00; A61M 2205/3592; A61M 2250/00; A61M 2205/3553; A61M 2202/0468; A61M 2205/33

(57) ABSTRACT

The invention relates to the field of public health and protection of the population against the spread of epidemics and pandemics and represents an alternative to quarantine measures and vaccination. Made and maintained in places where infection is possible are aerosol protective clouds which comprise a pathoprotector, i.e., a substance that precipitates on surfaces, including the mucosa, and prevents pathogens from entering body. Metal ion-based (such as silver ion-based) aqueous compositions are used as the pathoprotector. The compositions are safe and harmless to humans or animals, but can kill pathogens, preventing them from penetrating body tissues, reproducing and proliferating. Consistent application of the invention within population groups, cities, countries, and transportation systems makes it possible to arrest the spread of pathogens and to provide control over the process of inducing herd immunity in society by controlling the basic reproduction number of infections.

The invention relates to health protection, as well as protection of the public from epidemics and pandemics, including those transmitted by airborne droplets. Any practically significant tool for the society's fight against epidemics and pandemics must meet four basic requirements:
1. Security.
2. Efficiency.
3. Prompt response to emerging challenges.
4. Ethical, organizational, and economic potential for use.

Known means, closest to the specified requirements, are vaccines and sera. Their advantage is the great accumulated experience and traditions of development and implementation, the possibility of obtaining a reliable long-term strategic effect based on the development of immunity both at the level of individuals and groups, and society as a whole. Their disadvantage is a narrow range of action, long time that may pass until they are available for broad use since their development is started. Another problem is that neither their efficiency, nor the expediency of the time and money spent on their creation can be guaranteed.

Well-known effective means of countering epidemics and pandemics are sets of sanitary and epidemiological measures. For example, the active use of disinfectants, personal protective equipment, maintaining social distance, limiting contacts and mobility of the people, and other kinds for quarantine measures. Disinfectants are potentially hazardous to humans and the environment. Quarantine measures cause significant moral and economic harm to society. The general disadvantage of these measures is the lack of effectiveness against certain types of pathogens, high associated moral and material costs.

The use of sera and vaccines refers mainly to medical methods of combating epidemics and pandemics, while sanitary and epidemiological measures refer to administrative and organizational methods. The method proposed by the invention expands and supplements the existing limited range of methods by using technical means, predominantly electronic means. Disclosed herein is a technology for controlling and stopping infections by generating and maintaining protective aerosol clouds in places of possible contact with a pathogen and using ion-metal aqueous compositions (IMAC) as an active anti-pathogenic substance. This technology meets the four basic requirements listed above. The method and device of invention are suitable for use as a practical means of combating epidemics and pandemics, which is technologically, economically, organizationally accessible and has a wide range of action.

The essence of the invention is as follows: aerosol protective clouds (APO) of anti-pathogen protector are formed and maintained in places of possible contact with infected individuals. Anti-pathogen protector is a substance based on sprayable ion-metal aqueous compositions (IMAC), that prevents the penetration of the pathogen into the human body. Ideally, IMAC contains only water molecules and metal ions and do not contain any other additives. The closest known substances to IMAC are aqueous solutions of ionic silver [1] and other metals, present in water in ionic form. An anti-pathogen protector based on IMAC, in accordance with the rules and recommendations below, is safe, does not harm a person, particularly his mucous membranes, but is harmful to pathogens, prevents their invasion into body tissues, as well as their reproduction and spread [2].

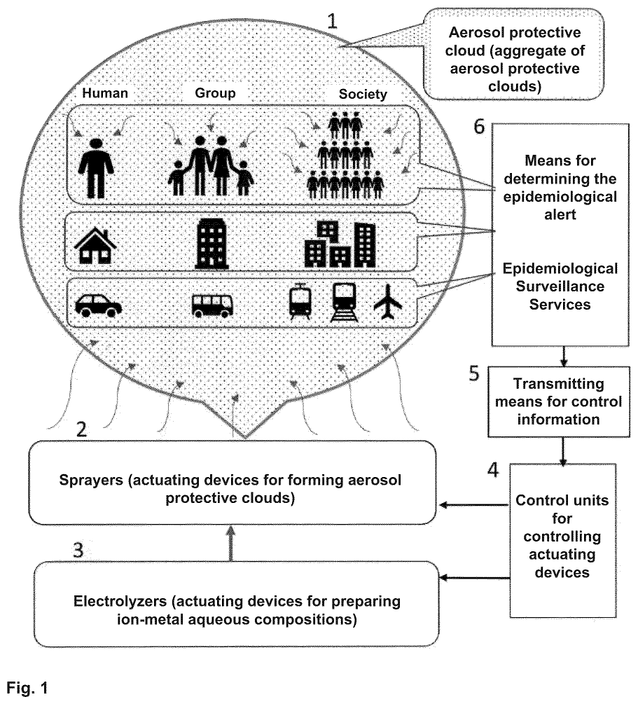

The concept in general is illustrated in FIG. 1. Aerosol protective clouds 1 of IMAC are generated and sustainably maintained in places potentially risky in terms of contamination. Aerosol particles of IMAC are present in the air of the protected volume in the form of mist, and also settle on surfaces, including the surfaces of human and/or animal mucous membranes, forming thereon a stable anti-pathogen protector film. Hereby, the pathogens are negated, both those suspended in the air due to contact with aerosol droplets, and those settled on surfaces due to contact with the IMAC film formed on these surfaces as a result of sedimentation of APO particles, including pathogens that get on the surfaces of the mucous membranes of vision, organs breathing, oral cavity, skin surface, food, clothes, other objects with which tactile contact is possible.

Broad geographical coverage and coordinated use of the method and devices of invention within social groups, settlements, cities, countries, as well as logistics transport systems allows protecting individuals or local social groups from infection. Moreover, it allows conducting a controlled process of acquiring herd immunity within a whole society by controlling and managing the contagion factor.

When epidemics and pandemics are emerging or are about to emerge, it is possible to control the activity of the pathogen by varying the composition and concentration of IMAC and the operational intensity of the aerosol devices that create and maintain protective clouds of the anti-pathogen protector. It is thereby possible to set and maintain the contagion factor in such a way that the society can, on the one hand, contact with pathogen under control and acquire a stable herd immunity to infection, and on the other hand, eliminate the risk of moral and economic losses unacceptable for society.

### TECHNIC OF IMPLEMENTING THE INVENTION

In order to control and manage the contagion factor, protective equipment (which can be stationary, mobile, or personal) is used in the form of sprayers (actuating devices that form aerosol protective clouds 2 of the active substance also known as pathoresist, based on IMAC) in places of potential risk of contagion by a pathogen. The effectiveness of the APO is adjusted by controlling the operating mode of the sprayers, i.e. changing their work intensity and/or the size of the generated aerosol particles, and/or controlling the operating mode of electrolyzers (actuating devices for preparing IMAC) by changing the composition of metal ions and/or their concentration in IMAC 4.

When it appears necessary to protect the public from infections, as well as to form herd immunity to a pathogen, the recommended parameters for the operation of APO systems are set by health authorities based on monitoring the situation, forecasts and statistical data 6. The parameters are transmitted by transmitting means for control information 5, for example, through the media, specialized means of communication, means based on IT-technologies, to manual or automatic control units for controlling actuating devices (sprayers and electrolyzers) 4.

A significant factor for the practical implementation of the method and device is the active substance (anti-pathogen protector) IMAC, its composition, dosage, method of preparation. Ion-metal aqueous composition is an aqueous solution of at least one metal present in the aqueous medium in ionic form, the solution lacking any sensible color, smell, taste. Preferable for the preparation of IMAC, is water with the lowest possible content of natural salts (salts of calcium, calcium, magnesium, sodium) as well as other impurities and suspensions. The best option in terms of efficiency and stability of the preparations is the use of distilled water or demineralized water, for example, by reverse osmosis. The mass fraction of initial water in IMAC is 99.5% or more. The mass fraction of metal ions is 0.5% or less. The method of introducing metal ions into water is of no substantial importance. However, the method of electrochemical dissolution shall be regarded as preferable, in view of high controllability and metrological accuracy of the process. The metals used for the preparation of IMAC can be silver, copper, gold and other metals, used both individually and in combination, depending on the type of pathogen and its characteristics. Thus, bacterial and viral pathogens are most sensitive to silver and gold ions, while fungal pathogens are most sensitive to copper ions. The optimal concentration of metal ions in IMAC used for the production of APO is 1 to 50 milligrams per liter. At lower concentration values, the effectiveness of the preparations is significantly reduced, whereas at higher values the increase in efficiency is insignificant but followed by essential increase in technical and economic costs.

The physical basis and principles of the way IMAC impact on pathogens lies in the ability of metal ions to form stable compounds with proteins, forming mainly albumins (simple water-soluble proteins). When a metal ion joins a protein, it releases some of the water molecules surrounding it and retained by it, forming on the protein surface a layer of impurity-free water—the strongest natural solvent with a dielectric constant of 81. This leads to the destruction of proteins. Proteins that have undergone destruction, as well as those that have reacted with metal atoms, are transformed and cease to perform their biological function. Metabolism and reproduction of pathogens that come into contact with IMAC are disrupted. At the same time, aqueous solutions containing metals in ionic form do not have a significant negative impact on the functioning of human organs and tissues. Metal ions do not have the ability to penetrate deep into tissues; their action is limited to the surface layer of tissue cells that is constantly regenerated in a natural way. Aqueous solutions of metal ions are unstable. Metal ions tend to, and gradually transform into a stable metallic form, while the anti-pathogenic effectiveness of the solutions decreases. The stability of ion-metal solutions and the rate of their deactivation are mainly influenced by two factors: the amount and composition of impurities contained in the raw water and the effect of light. Upon receipt of IMAC based on drinking water with a content of natural salts (salts of calcium, magnesium, sodium, potassium) at the level of several hundred milligrams per liter, the time for reducing the activity of monometallic silver IMAC by half is 1-3 days; when exposed to light—from a few tens of minutes to several hours. When using as a base demineralized water with an impurity content of less than 2-5 milligrams per liter, the time for reducing the activity of the preparation by half can be several weeks, and when exposed to light, a few tens of hours. The most stable IMACs are those prepared on the basis of water bidistillate. Metals that have passed from the ionic form to the metallic form lose their activity and, in the absence of conditions for secondary ionization, for example, by introducing additional reagents into the preparation (which is unacceptable within the method of invention), become biologically inert.

Sufficiently studied metals, the ions of which have the necessary antimicrobial, antifungal, antiviral properties and allow contact with human tissues with sufficiently high values of maximum allowable concentrations (MACs), include copper, silver, gold, iron, zinc, magnesium, etc. Perhaps, as we study properties, ionic solutions of other metals might be used.

Typical necessary and sufficient concentrations of metal ions in water to achieve the goals are concentrations from a few milligrams per liter to tens of milligrams per liter.

APO acts in the following way. Aerosol particles of IMAC APO, in addition to direct interaction in the air with aerosol particles containing a pathogen, also settle in the form of a protective film on the surfaces of the mucous membranes of the respiratory tract, eyes, oral cavity, on the skin of a person, his food, cutlery, clothing, household items and other surfaces with which he may have tactile contact. The protective film of IMAC that appears on surfaces as a result of deposition of aerosol particles reduces the activity of bacterial, fungal and viral pathogens that come into contact with it. For viral infections, when IMAC settles on mucous membranes, a double barrier is created that prevents the invasion of viruses into tissue cells. Firstly, disrupted is the structure of external protein formations (thorns) of viruses which help the virus entering the cell by interacting with the protein receptors of the cell. Secondly, the structure of protein receptors of the cells in the upper layers of tissues are transformed, thus further keeping the viruses from invasion of their genetic material into cells.

IMAC films formed on the surfaces of objects and mucous membranes as a result of exposure to APO have a durable effect. Their activity persists for some time after the cessation of APO. The typical aftereffect time of IMAC films ranges from a few minutes to a few tens of minutes. A person who has been exposed to APO for the time sufficient for the formation of surface films and left the area where APO is active, remains under protection for some time.

The technical means for implementing the method should primarily include devices for generating aerosol protective clouds: sprayers (actuating devices for the formation of aerosol protective clouds).

The hardware for implementing of the technology includes sprayers, both specially designed for the implementation of the method and well-known, widely available sprayers, preferably with a mechanical (including ultrasonic) principle for producing aerosol. These may be household devices (for example, devices and systems for controlling air humidity indoors [3] and industrial devices [4]. Generally, the devices contain a source of a sprayed substance (IMAC), a spray element and the means for the formation of a volumetric aerosol cloud (for example, fans, guide shutters and deflectors, nozzle distribution systems).

The optimal size of aerosol droplets generated by the sprayer depends on the tasks to be solved. It is known that the following value of the droplet size present in the air at the level of the respiratory organs for the delivery of an aerosol into the human respiratory system, shall be regarded as optimal: 0.5-2 microns for the lower sections, 2-5 microns for the middle sections and 5-50 microns for the upper sections. In practice, particles with a size of 50-100 microns and more do not penetrate into the respiratory organs and are generally optimal for the formation of protective films of the pathoresist on the mucous membranes of the eyes, mouth, skin, food and surrounding objects. In practice, droplets smaller than 0.5 microns in the respiratory system do not linger and remain suspended in the air for a long time, contributing to the active neutralization of the pathogen present in the air in the form of mist. It should also be noted that in many cases it is expedient to impart an electrostatic charge to the droplets of IMAC during spraying. This additionally intensifies the process of clearing the air from pathogens in the mist due to the action of the Coulomb forces of attraction.

The method of saturating the raw water with metal ions in devices implementing the current method of protection against the spread of infections, is not of substantial importance, however, aerosol devices specially created for the indicated purposes shall be regarded as optimal technical means of supporting the technology. The devices include as an integral part an assembly for preparing IMAC in tempore (immediately before spraying, without long-term intermediate storage of the preparation in transport or buffer containers), wherein IMAC is obtained using the principle of controlled electrolytic dissolution of metal electrodes under the action of electric current.

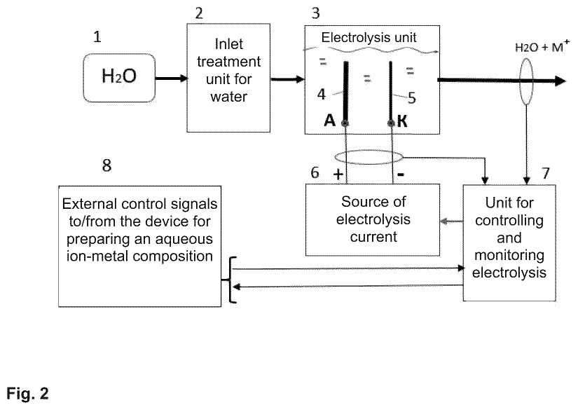

A schematical embodiment of a device (electrolyzer) for the electrolytic dissolution of metal electrodes induced by electric current is shown in FIG. 2 and consists of the following parts:

Primary source 2.1 of water supply, e.g., piped water. The inlet treatment unit 2.2 for raw water provides for the compliance of the parameters of the water coming from the primary source with the requirements of subsequent technological operations, in particular, for removing, where necessary, the excess content of salts and foreign impurities dissolved in water, for example, by reverse osmosis. Electrolysis unit 2.3 containing a cathode 2.5 and a sacrificial anode 2.4 made of a metal to be dissolved in water in the form of ions. To create an ion-metal aqueous composition containing several metals, anodes made of alloys [5] or several anodes made of various metals can be used, while there can also be several sources 2.6 of electrolysis current, wherein the ratio of metal concentrations in the resulting IMAC will be determined by the operating modes of the sources, the modes being set by a unit 2.7 for controlling and monitoring the electrolysis process, for example, made according to patents of the Russian Federation [6, 7, 8] and, as an option, having the ability to receive and transmit external control signals 2.8 between the device for preparing an aqueous ion-metal composition and the upper circuits for controlling the equipment.

Generally, it is advisable to implement IT technologies in the actuating devices (sprayers and electrolyzers) to adapt them for remote access to controlling the devices from health authorities for effective work as part of the healthcare system to control the spread of epidemics and pandemics, as well as to solve the problems of developing group immunity.

Safety, efficiency, technical, organizational and economic availability of the method consist of the following components:

There are approved standards for maximum permissible concentrations (MPCs) that are safe for the human body, for some metals applicable for practical use in the claimed method and device. Thus, according to some current standards [9], the MPC of silver and copper in drinking water is up to 0.1 mg. per 1 dm³ (liter) and up to 2.0 mg. per 1 dm³, respectively. The average daily human consumption of drinking water is 1.5 dm. Accordingly, 0.15 ml of silver and 3.0 ml of copper per day or 1.05 ml of silver and 21 ml of copper per week can enter the human body through the digestive system without harm to health. In the face of a high infectious danger and the need to reduce the contagion factor by 3 or more times, a solution containing 10 ml per 1 dm³ of silver can be used for the preparation of IMAC and APO may be of high density with a content of 10 ml of aerosol per 1 m³. If a person remains in the APO coverage area for 50 hours a week (work, transport, shops), up to 3 mg of silver per week can enter his body through the respiratory system. It is assumed that the volume of air passing through the respiratory system of a person during moderate physical exertion is 1 m³ per hour, and the absorption coefficient of aerosol mist in the respiratory system is 0.6. Thus, cumulative doses of silver metal that can enter the human body under action of the proposed method and device through the respiratory system are commensurate with the MPC values established by the standards as safe when the metal enters the body through the digestive system. Given that IMAC solutions contain nothing extraneous except purified water and metal ions, and metal ions do not have the ability to penetrate deeply into tissues, and the fact that continuing rejuvenescence takes place in the respiratory system, wherein mucous secretions with substances and dust particles settled thereon are excreted from the body, solutions of IMAC delivered to the body by means of aerosol particles and in the quantities indicated above, necessary and sufficient for stopping the acute phases of epidemics and pandemics, cannot harm the human body. When copper is used in IMAC, the allowable content of this metal can be an order of magnitude more than the content of silver.

It should be noted that the presence of ionic silver in IMAC is desirable in all cases. Ionic silver has a positive effect on the overall course of the disease, reduces the likelihood of hyperreactions and a cytokine storm of the immune system.

A potential increase of 5-25% relative humidity in rooms where APO is used may be regarded as hygienic disadvantages of the method and device. This may limit the possibility of using the method and devices in areas with tropical climate and in temperate climate zones in the summer, in rooms and vehicles not equipped with air conditioning systems with high initial temperature and air humidity.

To date, due to the development of related industries, there is a fleet of equipment which can be used as actuators functioning as IMAC sprayers. Devices and systems for air humidification and devices for generating water mist of various classes and purposes can be used as sprayers with suitable parameters of the produced aerosol, both low-capacity devices designed to serve the needs of one person, and high-capacity industrial installations capable of processing significant volumes and areas.

In case of remote centralized production of IMAC, separately from sprayers, suitable are technologies, equipment, infrastructure, logistics networks of companies producing drinking water. The average daily requirement for IMAC per person in the acute phases of the development of epidemics and pandemics is 0.1-0.3 liters per day, i.e., 5-15 times less than the average daily consumption of drinking water (1.5 liters). Large-scale production of IMAC is possible on the basis of available technologies, basic equipment and production capacities. To meet the demand the companies producing drinking water will need readjustment of some equipment. The increase in output and load factor will not exceed 25%, i.e., not beyond typical market fluctuations and technological capabilities of the industry.

The production costs of IMAC are determined mainly by the costs of preparing the raw water. The production of demineralized water is technologically simpler and less costly than the production of drinking water and, taking into account the costs of electrolysis and the consumption of metals, the total costs for the production of IMAC do not exceed the costs for the production of drinking water. The industry average cost of production of drinking bottled water in the Russian Federation using returnable packaging and existing logistics is currently 6-12 rubles. (USD 0.08-0.17) per 1 liter. When using electrolyzers built into sprayers and using a water main as a primary source of water, the cost of IMAC decreases many times over.

The declared properties and parameters of the method and device are confirmed by experimental testing of the virucidal properties of ion-metal aqueous compositions VAH-50 manufactured by OOO New Line, St. Petersburg, conducted in the laboratory of viral infections of the Federal Service for Supervision of Consumer Rights Protection and Human Welfare in Saint Petersburg.

The study used the influenza virus (“swine flu”) A/Puerto Rico/8/34 (H1N1) from the collection of viral strains of the Saint-Petersburg Pasteur Institute. White mature mice (females) of the Balb/c line weighing 16-18 g (age 5-6 weeks) with a total number of 150 individuals were used as experimental animals. The studies were carried out in accordance with the guidelines and documents [10, 11]. The purpose of the studies was to confirm the virucidal efficacy of the doses of IMAC aerosols intended for practical use and to identify side effects, if any.

The animals were kept in the atmosphere of the aerosol on test. Three groups were used. Mice in groups 0 and 1 were infected intranasally under light ether anesthesia with the virus at a dose of 3×102 TNC50 per mouse in a volume of 30 μL. Mice in group 2 were not infected with the virus but were exposed to the maximum technologically available dose of IMAC aerosol for the equipment produced by New Line. Group 2 was used to identify side effects of the preparations and technology. Controlled parameters were the dynamics of changes in the weight of animals during the studies and the size of the foci of post-influenza pneumonia, which was expressed as a percentage of the total surface of the lungs. The sizes of the foci were determined visually at the end of the experiment, euthanasia and dissection of the animals. Comparisons between groups were made using Student's t-test. Differences between groups were considered significant if the p parameter did not exceed 0.05.

The results of using an aerosol of ion-metal composites against the Rico/8/34 (H1N1) virus are presented in Table 1.

TABLE 1

| | | Average size | |
|---|---|---|---|
| | | of foci of | |
| Group | Experiment conditions | pneumonia, % | p |
| 0 | Infected animals without aerosol | 28.3 ± 5.1 | 1.000 |
| | treatment | | |
| 1 | Infected animals, therapeutic | 10.0 ± 2.5 | 0.005 |
| | (technologically accepted as | | |
| | nominal) dose of aerosol treatment | | |
| 2 | Non-infected (intact) animals, | 0.0 ± 0.0 | <0.0001 |
| | extremely high (technologically | | |
| | possible maximum) dose of aerosol | | |
| | treatment | | |

The conducted tests studying the projective properties of the aerosol of ion-metal composites in experiments in vivo, using the model of experimental influenza pneumonia in white mice confirmed the presence of virucidal properties of the studied preparation produced and used according to the technology of invention. The experiment has shown 2.83 times reduction in the area of pneumonia foci. At extremely high doses of aerosol in the surrounding atmosphere in the lungs of intact animals no visually detectable pathological foci of lung tissue lesions were formed. The dynamics of changes in the weight of animals in the experiment does not deviate from the typical dynamics of healthy animals not exposed to external factors. Thus, no side effects were found in the experiment.

It should be noted that the design of the experiment included a targeted study of the virucidal activity of only that part of the APO IMAC, which was formed and maintained in the form of an anti-pathogen protector film on the surfaces of the mucous membranes of the respiratory system during a massive one-time attack of a large-drop pathogen-containing spray. At present, the effectiveness of other channels of acting upon the pathogen by the APO remains unexplored, for example, the acting through direct contact of APO particles with pathogens suspended in the air; the acting through the weakening of the pathogen due to the anti-pathogen protector films formed on the mucous membranes of the infection donor; the acting through tactile contacts with surfaces (including those of food) covered with anti-pathogen protector.

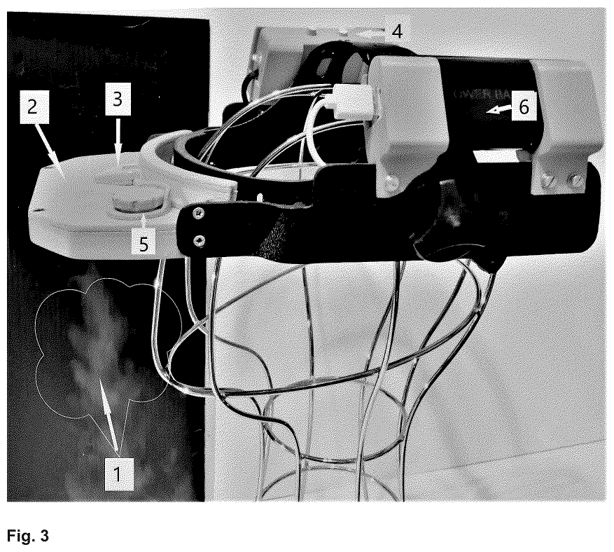

One example of a hardware implementation of the method and device is a portable device that performs the function of personal protective equipment (PPE) against airborne infections is shown in FIG. 3. The device protecting the most vulnerable area of the human body: his face (mucous membranes of the eyes, mouth, respiratory organs), is designed in the form of a headdress with a visor. The name of the device is Sputnik-6, the manufacturing company is OOO New Line, St. Petersburg, Russia. The device forms and maintains APO IMAC 1 in the area of the user's face. It contains an actuating device for the formation of an aerosol protective cloud, i.e., a sprayer 2, an actuating device for preparing an aqueous ion-metal composition, i.e. an electrolyzer 3, a control unit 4 for controlling actuating devices, a filling tank 3.5 for raw water, and battery 3.6 for powering the device. The control unit 4 is provided with manual controls in the form of two buttons for controlling the operation mode of the sprayer: on/off button and therapeutic/intensive button for switching the intensity of the APO spray (dose). The user, having received, for example, through his organs of hearing or vision, information about the presence of an epidemiological threat, for example, from epidemiological surveillance services or guided by their own observations and assessment of the situation, activates the device and sets the operating mode in accordance with the level of danger. The PPE device “Sputnik-6” is capable of continuous autonomous operation without refilling with raw water and recharging or changing the battery for at least 5-10 hours. Alternatively, the device can be filled with prefabricated IMAC. The weight of the fully equipped device is not exceeding 250 grams.

Based on the data obtained in the course of creation and testing of the invention, there is reason to believe that wide and diverse application of the method and device of invention allows to controllably reduce the contagion factor of viral infections. When the infection is at the plateau stage, and the intensity of use is moderate the contagion factor may decrease 1.0-1.5 times, whereas in peak situations and high intensity of use the contagion factor may decrease three-fold or more.

With regard to cumulative criteria of safety, efficiency, technical, organizational and economic parameters considered in conjunction, the method and device for stopping epidemics and pandemics are appropriate and available to the society for practical use.

### BIBLIOGRAPHY

[1] Kulsky L. A. Silver water—Kyiv: Naukova Dumka, 1968.
[2] Konychev A. V., Koreshkina T. A., Kokorin K. V. The use of ionic silver solutions in the treatment of purulent diseases//Abstracts of the All-Russian Conference “Actual problems of operative surgery, surgical anatomy and pathophysiology”.—St. Petersburg, MALO.—1999—page 98.
[3] https://www.ixbt.com/home/humidifier-guide-2019.html
[4] https://air-rus.ru/catalog/promyshlennye-uvlazhniteli/[5]
[5] https://aquazip.ru/[6]
[6] Patent RU 2262486.
[7] Patent RU 2145941.
[8] Patent RU 2060958.
[9] https://ochistkavodi.ru/baza-znanij/sanpin-i-pdk/sanpin.html
[10] Rules of Laboratory Practice in the Russian Federation (Order of the Ministry of Health of the Russian Federation No. 708n dated Aug. 23, 2010).
[11] Guidelines for Conducting Preclinical Studies of Preparations, Part one//ed. A. N. Mironova (2012).

1-6. (canceled)

7. A method for arresting epidemics and pandemics, the method comprising making and maintaining an aerosol protective cloud of a pathoprotector in places of potential contact with a pathogen carrier.

8. The method according to claim 7, wherein metal-ion water system is used as the pathoprotector.

9. The method according to claim 8, wherein used as the metal-ion water system are aqueous solutions of silver and other metals present in water in an ionic form, the aqueous solutions being harmless for humans but fatal for pathogens thus preventing the pathogens from invading body tissues, and from reproducing and transmitting.

10. The method according to claim 7, wherein the aerosol protective cloud is made by a sprayer.

11. The method according to claim 10, wherein parameters of the aerosol protective cloud are changed in the sprayer subject to epidemiological situation at the place of making the aerosol protective cloud.

12. The method according to claim 9, wherein the aqueous solutions of metals are prepared by electrolysis.
